(19) United States
(12) Plant Patent Application Publication
(10) Pub. No.: US 2002/0188979 P1
(43) Pub. Date: Dec. 12, 2002

(54) CHRYSANTHEMUM PLANT NAMED 'GEDI ONE AVI'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,493

(22) Filed: Jun. 4, 2001

(51) Int. Cl. A01H 5/00

(52) U.S. Cl. PLT/289

(57) ABSTRACT

A new and distinct Chrysanthemum plant variety characterized by a Large decorative type inflorescence, consistent flowering response to short days, blooming consistently after 49 days of short day length, free branching habit, and consistent natural season flowering habit during the first two weeks of September.

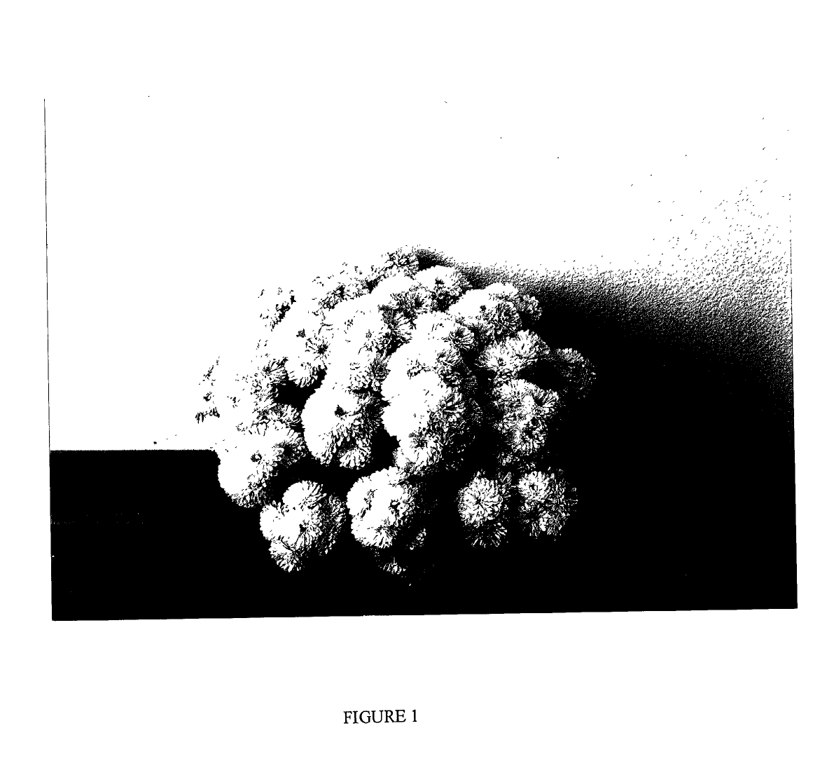

### BACKGROUND OF THE INVENTION

The present invention relates to a new and distinct cultivar of Chrysanthemum plant, botanically known as Dendranthema morifolium and herein after referred to by the cultivar name ‘GEDI ONE AVI.’

This new cultivar originated from hybridization of a female Chrysanthemum plant cultivar ‘Pinos’ (U.S. Plant Pat. No. 10,208) with a male Chrysanthemum plant cultivar ‘Dark Veria’ (U.S. Plant Pat. No. 10,216). The new cultivar was discovered by the inventor in October 1997.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE AVI’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE AVI.’ These characteristics in combination distinguish ‘GEDI ONE AVI’ as a new and distinct Chrysanthemum:

1. Large decorative type inflorescence;

2. Consistent flowering response to short days, blooming consistently after 49 days of short day length;

3. Free branching habit; and

4. Consistent natural season flowering habit during the first two weeks of September.

Plants of the new cultivar are similar to plants of the parent cultivar, ‘Pinos,’ in most horticultural characteristics, however plants of the new cultivar differed from plants of the cultivar ‘Pinos’ in the following characteristics:

1. The new cultivar has a wider diameter flower;

2. The new cultivar has many more ray florets than the parent; and

3. The new cultivar is taller and wider.

Plants of the new cultivar are similar to plants of the parent cultivar, ‘Dark Veria,’ in most horticultural characteristics, however plants of the new cultivar differed from plants of the cultivar ‘Dark Veria’ in the following characteristics:

1. The new cultivar has a wider diameter flower;

2. The new cultivar has many more ray florets than the parent;

3. The new cultivar naturally blooms three weeks earlier; and

4. The new cultivar is taller and wider.

The new cultivar can be compared to the Chrysanthemum cultivar ‘CLEAGAR GF1’ (disclosed in Plant Pat. No. 10,463). However plants of the new Chrysanthemum differed from the plants of the cultivar ‘CLEAGAR GF1’ in the following characteristics:

1. New cultivar naturally blooms 2 weeks earlier;

2. New cultivar has larger diameter blooms;

3. New cultivar has more blooms per flowering branch; and

4. New cultivar is taller and wider.

### BRIEF DESCRIPTION OF THE PHOTOGRAPHS

The photograph shows a plant of ‘GEDI ONE AVI’ grown in a 6-inch container. A single cutting was used in the container.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE AVI’

Commercial classification: Garden-type Chrysanthemum

Parentage: Hybridization of female parent ‘Pinos’ (U.S. Plant Pat. No. 10,208) with male

parent ‘Dark Veria’ (U.S. Plant Pat. No. 10,216)

### Propagation

Time to Rooting: 7 to 14 days at approximately 21° C.

Rooting Habit: Fine, fibrous

### Plant

Growth Habit: Mounding herbaceous perennial

Height: Approximately 27.5 cm

Growth rate: Moderate

Branching Characteristics: Free Branching

Length of Lateral Branches: Approximately 23 cm

Number of leaves per lateral branch: Approximately 26

Stem color: Near 138A

### Foliage

Leaf:

Arrangement.—Alternate single

Average length.—Approximately 5.6 cm

Average width.—Approximately 3.4 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color.—young foliage upper side: Near 147A young foliage under side: Near 147B mature foliage upper side: Near 147A mature foliage under side: Near 147B venation upper side: Near 147B venation under side: Near 147B

Petiole:

Average Length.—Approximately 2.0 cm

### Bloom

Inflorescence:

Flowering habit.—Induced by darkness period greater than 13.5 hours, approximately 49 days of appropriate day length required to induce and develop blooms

Inflorescence form.—Decorative

Natural flowering season.—Approximately the first two weeks of September

Number of inflforescences per stem.—Approximately 12

Inflorescence diameter.—Approximately 5.3 cm

Inflorescence depth.—Approximately 2.0 cm

Inflorescence longevity on plant.—Approximately 22-24 days

Persistence.—Persistent

Ray Florets:

Appearance.—Matte

Texture.—Smooth

Average number per flower.—Approximately 190

Shape.—Cylindric

Margin.—Entire

Apex.—Obtuse

Length.—Approximately 1.8 cm

Width.—Approximately 5 cm

Color.—upper surface at first opening: Near 14A upper surface at maturity: Near 7B upper surface atfading: Near 7C under surface atfirst opening: Near 14B under surface at maturity: Near 7C under surface atfading: Near 7D

Disc florets:

Appearance.—Shiny

Texture.—Smooth

Average number per flower.—Approximately 35

Shape.—Cylindric

Apex.—Obtuse

Average length.—Approximately 0.3 cm

Average Width.—Approximately 0.1 cm

Color.—at first opening: Near 21A at maturity: Near 15A at fading: Near 1 3A

Peduncle:

Length.—at terminal end (shortest): Approximately 2.3 cm at lateral end (longest): Approximately 3.5 cm

Strength.—Moderate

Color.—Near 146A

Habit.—Erect

Inflorescence bud:

Length.—Approximately 0.5 cm

Diameter.—Approximately 0.5 cm

Form.—Globular

Color.—Near 12A

Sepals:

Appearance.—Matte

Texture.—Lightly pubescent

Number.—Approximately 30 to 40

Shape.—Ovate

Margin.—Entire

Apex.—Acute

Color.—upper side: Near 137B under side: Near 137B

### Reproductive Organs

Ray Florets:

Pistillate.—Number of pistils perflower: 1 Stigma shape: 2 branched Stigma color: Near 15A Stigma Length: Approximately 0.1 Style color: Near 144B Style length: Approximately 0.3 cm

Disc Florets:

Pistillate.—Number of pistils per flower: 1 Stigma shape: Cylindric Style length: Approximately 0.1 cm

Staminate.—Number ofstamens per flower: Approximately 5 Anther shape: Tubular Anther color: Near 15A Pollen color: Near 15A

### Other Characteristics

Disease Resistance: Neither resistance nor susceptibility observed

‘GEDI ONE AVI’: Table of R.H.S. Color Codes

| | | | |
|---|---|---|---|
| FOLIAGE | Leaf | young foliage upper surface | Near 147A |
| | | young foliage under surface | Near 147B |
| | | mature foliage upper surface | Near 147A |
| | | mature foliage under surface | Near 147B |
| | | venation upper surface | Near 147B |
| | | venation under surface | Near 147B |
| BLOOM | Ray Florets | upper surface at first opening | Near 14A |
| | | upper surface at maturity | Near 7B |
| | | upper surface at fading | Near 7C |
| | | under surface at first opening | Near 14B |
| | | under surface at maturity | Near 7C |
| | | under surface at fading | Near 7D |
| | | Pistillate: Stigma | Near 15A |
| | | Pistillate: Style | Near 144B |
| | Disc Florets | at first opening | Near 21A |
| | | at maturity | Near 15A |
| | | at fading | Near 13A |
| | | Staminate: Anther | Near 15A |
| | | Staminate: Pollen | Near 15A |
| | Peduncle | | Near 146A |
| | Inflorescence | | Near 12A |
| | Bud | | |
| | Sepals | upper surface | Near 137B |
| | | under surface | Near 137B |

What is claimed is:

1. A new and distinct variety of Chrysanthemum plant named ‘GEDI ONE AVI’ as herein illustrated and described.
